(19) United States
(12) Patent Application Publication
(10) Pub. No.: US 2015/0313672 A1
(43) Pub. Date: Nov. 5, 2015

(54) IDEAL VALUES FOR LASER PARAMETERS FOR CALCULI REMOVAL

(71) Applicant: Cook Medical Technologies LLC, Bloomington, IN (US)

(72) Inventors: Thomas Milner, Austin, TX (US); Joel Teichman, Vancouver (CA)

(73) Assignee: Cook Medical Technologies LLC, Bloomington, IN (US)

(21) Appl. No.: 14/699,687

(22) Filed: Apr. 29, 2015

Related U.S. Application Data
(60) Provisional application No. 61/986,446, filed on Apr. 30, 2014.

(51) Int. Cl. A61B 18/24

(52) U.S. Cl. CPC A61B 18/245; A61B 2018/00505

(57) ABSTRACT

A set of ideal values for laser parameters is provided for a laser system for the fragmentation and removal of calculi. Particularly, the ideal values for laser parameters increases the effectiveness of removing calculi by increasing the pulse width. This set of ideal values for laser parameters increases the effectiveness of calculi removal by decreasing retropulsion distance of calculi fragments and by breaking up the calculi into smaller fragments that can then be easily removed from the body without the use of baskets or graspers.

### CROSS-REFERENCE TO RELATED APPLICATIONS

This application claims priority from U.S. Provisional Application No. 61/986,446, filed on Apr. 30, 2014, the entirety of which is hereby fully incorporated by reference herein.

### TECHNICAL FIELD

This disclosure generally relates to the use of a laser system for the removal of unwanted materials such as calculi, deposits and tissue from body lumens. More particularly, this disclosure relates to ideal values for laser parameters used in a laser system to remove calculi from the body.

### BRIEF SUMMARY

Disclosed is a set of ideal values for laser parameters for use in a pulsed laser system for the fragmentation and removal of calculi. Particularly, the disclosed set of ideal values for laser parameters are for use in a laser system that uses a Ho:YAG laser. Particularly, the ideal values for laser parameters minimizes the amount of calculi movement while providing for calculi fragmentation by increasing the pulse width. Doing so reduces retropulsion distance that can cause trauma to the surrounding tissue and allows for the removal of the fragmented calculi through the voiding of the water or saline flow used during the procedure. The disclosed system can also be used on soft tissue procedures to remove polyps or tumor cells.

In one embodiment, an apparatus for fragmenting calculi comprises a source of laser pulses, an optical fiber having a distal end configured to be in close proximity with said calculi and a proximal end that is configured to receive laser pulses from said source of laser pulses when said optical fiber is operatively engaged with said source of laser pulses, and a source of laser pulses is configured to specifically generate laser pulses with an optical pulse width between 400 μs and 600 μs.

In another embodiment, the apparatus for fragmenting calculi comprises a source of laser pulses configured to specifically generate laser pulses with an optical pulse width between 600 μs and 1000 μs.

In another embodiment, the apparatus for fragmenting calculi comprises a source of laser pulses configured to specifically generate laser pulses with an optical pulse width of 438.8 μs.

In another embodiment, the apparatus for fragmenting calculi comprises a source of laser pulses configured to specifically generate laser pulses with an optical pulse width of 584.4 μs.

In another embodiment, the apparatus for fragmenting calculi comprises a source of laser pulses configured to specifically generate laser pulses with an electrical pulse width at least 1000 μs.

In another embodiment, the apparatus for fragmenting calculi comprises a source of laser pulses configured to specifically generate laser pulses with an electrical pulse width of 1000 μs.

In another embodiment, the apparatus for fragmenting calculi comprises a source of laser pulses configured to specifically generate laser pulses with an electrical pulse width of 1250 μs.

In another embodiment, a method for fragmenting calculi comprises providing a source of laser pulses, providing an optical fiber having a distal end configured to be in close proximity with the calculi and a proximal end that is configured to receive laser pulses from the source of laser pulses when the optical fiber is operatively engaged with the source of laser pulses, and calibrating the source of laser pulses to specifically generate laser pulses with an optical pulse width between 400 μs and 600 μs.

In another embodiment, the method for fragmenting calculi comprises a source of laser pulses configured to specifically generate laser pulses with an optical pulse width between 600 μs and 1000 μs.

In another embodiment, the method for fragmenting calculi comprises a source of laser pulses configured to specifically generate laser pulses with an optical pulse width of 438.8 μs.

In another embodiment, the method for fragmenting calculi comprises a source of laser pulses configured to specifically generate laser pulses with an optical pulse width of 584.4 μs.

In another embodiment, the method for fragmenting calculi comprises a source of laser pulses configured to specifically generate laser pulses with an electrical pulse width at least 1000 μs.

In another embodiment, the method for fragmenting calculi comprises a source of laser pulses configured to specifically generate laser pulses with an electrical pulse width of 1000 μs.

In another embodiment, the method for fragmenting calculi comprises a source of laser pulses configured to specifically generate laser pulses with an electrical pulse width of 1250 μs.

Other systems, methods, features and advantages of the invention will be, or will become, apparent to one with skill in the art upon examination of the following figures and detailed description. It is intended that all such additional systems, methods, features, and advantages be within the scope of the invention, and be encompassed by the following claims.

### BRIEF DESCRIPTION OF THE DRAWINGS

The accompanying drawings are included to provide further understanding of the claims, are incorporated in, and constitute a part of this specification. The detailed description and illustrated examples described serve to explain the principles defined by the claims.

FIG. 1 is a block diagram of an embodiment of the laser system used to generate the ideal values for laser parameters for calculi removal.

FIG. 2 is a flow chart of an embodiment of the laser system used to generate the ideal values for laser parameters for calculi removal.

FIG. 3 is a graph with electrical pulse width represented on the x-axis and optical pulse width represented on the y-axis wherein optical pulse width is shown as a function of electrical pulse length for a Ho:YAG laser.

FIG. 4 is a graph with electrical pulse width represented on the x-axis and optical pulse width represented on the y-axis, wherein retropulsion length is shown as a function of electrical pulse width for a Ho:YAG laser using two different fiber sizes.

FIG. 5 is a graph with electrical pulse width represented on the x-axis and crater volume represented on the y-axis, wherein crater volume is shown as a function of electrical pulse width for a Ho:YAG laser using two different fiber sizes.

FIG. 6 is a graph with electrical pulse width represented on the x-axis and

$$\frac{retropulsion distance}{ablation volume}$$

(“ideality”) represented on the y-axis, wherein ideality is shown as a function of pulse width for a Ho:YAG laser using two different fiber sizes.

FIG. 7 is a graph with electrical pulse energy represented on the x-axis and retropulsion length represented on the y-axis, wherein retropulsion length is shown as a function of electrical pulse energy for a Ho:YAG laser at two constant pulse widths.

FIG. 8 is a graph with electrical pulse width represented on the x-axis and crater volume represented on the y-axis, wherein crater volume is shown as a function of electrical pulse energy for a Ho:YAG laser at two constant pulse widths.

FIG. 9 is a graph with electrical pulse energy represented on the x-axis and

$$\frac{retropulsion distance}{ablation volume}$$

(“ideality”) represented on the y-axis, wherein ideality is shown as a function of electrical pulse energy for a Ho:YAG laser at two constant pulse widths.

### DETAILED DESCRIPTION OF THE INVENTION

Calculi can form in various parts of the body, such as the kidneys or the gallbladder, which can cause pain or damage to the body if not removed. Open surgical intervention was once the standard treatment for the removal of calculi, particularly when such calculi was located in a body lumen. However, less invasive techniques have emerged as safe and effective alternatives for the removal of calculi in the body.

Lithotripsy is a less invasive technique used to remove calculi in the body. It involves the crushing of the calculi into fragments that are easier to remove from the body. Lasers are often used as a power source for lithotripsy as the laser fiber is small and therefore the aperture of the working channel can be minimized. Laser systems may be used to break down calculi into smaller pieces. In particular, the laser system may be configured to generate and output a laser beam or other high concentrated beam of energy which may be transmitted to the treatment site. At the treatment site, the laser beam fragments, pulverizes or erodes the calculi.

Ho:YAG lasers may be used to break down calculi or stones into smaller pieces to facilitate removal of the calculi. The Ho:YAG laser can be used not only for the removal of calculi, but also for other soft tissue procedures. The Ho:YAG laser is typically transmitted through a fiber. When a Ho:YAG laser, after travelling the length of the fiber, is fired into a liquid medium the laser energy produces a vaporization bubble. The Ho:YAG laser produces a light at a wavelength of about 2.0 to 2.1 microns, depending on the precise formulation of the Ho:YAG rod, in a pulsed fashion. The Ho:YAG laser is effective because the aforementioned wavelengths are well absorbed by water and other liquid mediums. Further, all stones in a body lumen absorb this wavelength well, regardless of the stone color, because of the water in and on the surface of the stone. Although the various commercial models of Ho:YAG lasers vary slightly, the pulse duration of the Ho:YAG laser ranges from 250-350 μs, pulse energy from 0.2-4.0 J/pulse, frequency from 5-45 Hz and the average power from 30-80 W.

The dominant mechanism in Ho:YAG laser lithotripsy is photothermal along with the added minor effects of acoustic emission. Direct light absorption of the calculi increases the temperature of the irradiated volume above the ablation threshold, thereby causing the ejection of fragmented breakdown products. As well, absorption of laser energy by water between the stone and the fiber tip induces vapor bubble formation and collapse that generates shock waves. This laser-calculi interaction subjects the calculi to retropulsion forces induced by the combined effects of the ablated particle ejection, interstitial water vaporization, and bubble expansion and collapse. Therefore, the firing of each pulse causes the calculi to be displaced away from the fiber of the Ho:YAG laser.

Lasers rely on four main parameters for their performance—wavelength, spot size, pulse energy, and pulse width. As discussed earlier, the Ho:YAG laser produces a light at a wavelength of about 2.0 to 2.1 microns. The spot size of the laser is determined by the diameter of the fiber of the Ho:YAG laser, with a greater diameter achieving a greater spot size. Pulse energy determines the energy that the laser generates with each pulse. Pulse width, often referred to as pulse duration, can be quantified as either electrical pulse width or optical pulse width. Electrical pulse width is the time that the energy source to the laser is being pulsed. Optical pulse width is the time the laser light takes to exit the laser.

A clinical objective is to complete intracorporeal laser lithotripsy procedures as quickly as possible. To accomplish laser lithotripsy procedures quickly, experimental data has demonstrated that high pulse energy is better since it is faster at removing the calculi. However, retropulsion increases substantially at a higher pulse energy setting. Thus, a urologist might choose a high pulse energy setting to finish the case quickly as long as the potential need to chase the stone is acceptable. This strategy might be particularly useful for large bladder stones, for which stone volume requires lengthy operative time devoted to laser lithotripsy and the large stone mass has less retropulsion than a smaller stone. For instance, the use of high pulse energy would be much more effective on large kidney and bladder stones that have a sizes that fall on the range between 1-2 sonometers.

Conversely not all urologists would accept this trade-off of faster lithotripsy but with greater retropulsion, especially for ureteroscopy. Retropulsion implies increased and potentially wasted operative time spent chasing the stone. Retropulsion also implies less lithotripsy since the high pulse energy settings produce more retropulsion and less fragmentation. Moreover, the larger fragments produced by high pulse energy may require basketing and increase costs due to increased operative time and basket use. A strategy of low pulse energy to create tiny debris may produce slow lithotripsy but potentially may not be as inefficient when compared to the time spent chasing the stone and its fragments as a result of retropulsion. Particularly, the use of high pulse energy would be less effective on smaller calculi, such as ureteral stones, that are often between 6-7 millimeters in size. In smaller calculi, the stones are prone to more movement and therefore a strategy of low pulse energy is more efficient as it provides for less operative time and basket use.

The present disclosure describes a laser system that is configured to operate at a predetermined set of laser parameter values to output a laser beam that removes calculi with minimum retropulsion. Effectiveness is maximized by minimizing the movement of the calculi. The present disclosure describes a set of laser parameter values that minimizes retropulsion by maximizing the pulse width of the laser.

The embodiments described in this disclosure will be discussed generally in relation to application of ideal values of laser parameters of a laser system to fragment smaller calculi in lithotripsy, but the disclosure is not so limited and may be applied to other soft tissue procedures, such as the removal of polyps and tumors.

As used herein, the term “retropulsion” refers to the amount of stone migration in the body of a human or mammal being acted upon by a laser pulse. High retropulsion can cause trauma to the tissue and potential ureter perforation.

The term “ablation” refers to the volume of stone that is removed from the surface of the calculi.

The term “ideality” refers to the measure of effectiveness of the laser system at fragmenting and removing smaller calculi in the body. Ideality is measured as:

$$Ideality = \frac{Retropulsion Distance ( mm )}{Ablation Volume ( {mm}^{3} )}$$

The parameters of the laser are more effective the lower the ideality ratio is—(e.g. higher ablation volume and lower retropulsion distance).

The term “ideal values” refer to values for laser parameters that are adapted to effectively fragment and remove smaller calculi within the body.

In one aspect, the ideal values for the laser parameters at which the laser system is operated are chosen to reduce retropulsion distance. This reduces or eliminates any trauma caused by chasing the stone in the body or using baskets and graspers to remove stone fragments. Ideal values for the laser parameters are achieved by reducing the peak power of the laser by increasing the electrical pulse width and reducing the electrical pulse energy.

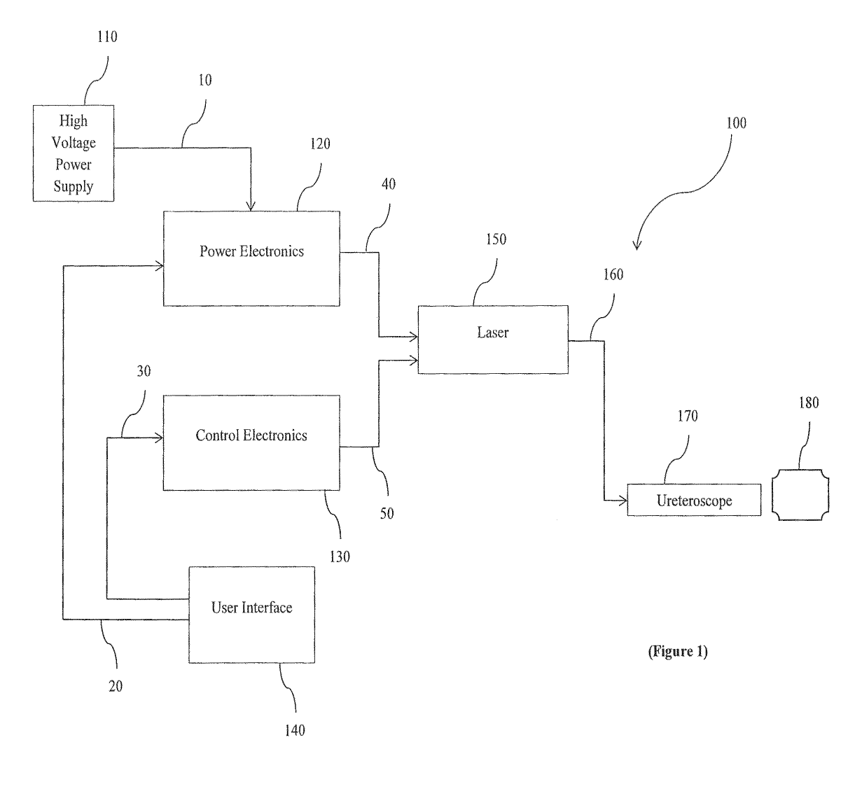

FIG. 1 illustrates a block diagram of an example embodiment of a laser system 100 configured to operate in accordance with the ideal values for laser parameters for calculi removal.

The laser system 100 includes a high voltage power supply 110, power electronics 120, control electronics 130, user interface 140, a laser 150, a fiber 160, and an ureteroscope 170. As shown in FIG. 1, the high voltage power supply 110 is connected through a high voltage connection 10 to the power electronics 120. The user interface 140 is connected to the control electronics 130 through a data connection 30. Similarly, the user interface 140 is connected to the power electronics 120 through a data connection 20. The laser 150 is connected to the control electronics through a command data connection 50 and to the power electronics 120 through a high voltage connection 40. The fiber 160 passes through the ureteroscope 170 at the distal end and the proximal face of the fiber 160 is connected to the laser 150. The energy from laser 150 is transferred through the fiber 160 and the ureteroscope 170 before it is fired into the liquid medium at calculi 180.

In operation, the laser system may be configured to operate in accordance with operating parameters to output a laser beam having corresponding characteristics to break down the calculi in a desired way. The ideal values for the laser parameters are entered or input into the user interface 140. At least some of the laser parameters are variable parameters such as electrical pulse width, optical pulse width, pulse energy, and/or pulse frequency. Upon receipt of the ideal values, the user interface 140 sends the ideal values through the data connection 30 to the control electronics 130. The control electronics 130 receives the ideal values from the user interface 140 and configures the laser parameters to output a laser with parameters having the ideal values. Similarly, the user interface 140 communicates with the power electronics 120 through the data connection 20 by sending the ideal values for laser parameters necessary to power the system. The power electronics 120 receives the signal from the user interface 140 and configures the laser parameter to the ideal value for power that will be generated when the laser is actuated.

In response to receipt of the ideal values from the user interface 140, the control electronics 130 sends signals to the laser 150 through the command data connection 50 so that, when activated, the laser 150 emits a laser pulse having parameters that correspond with the ideal values input through the user interface 140. The power electronics 120 provides the laser 150 with the amount of power indicated by the user interface 140 through the high voltage connection 40. The high voltage power supply 110 supplies the power electronics 120 with the power to power the laser 150 through the high voltage connection 10.

In further operation, the pulse generated from the laser 150 is transmitted through the fiber 160 to the treatment site where the calculi 180 is located. In one embodiment, the fiber 160 is a 365 μm fiber. In another embodiment, the fiber 160 is a 273 μm fiber.

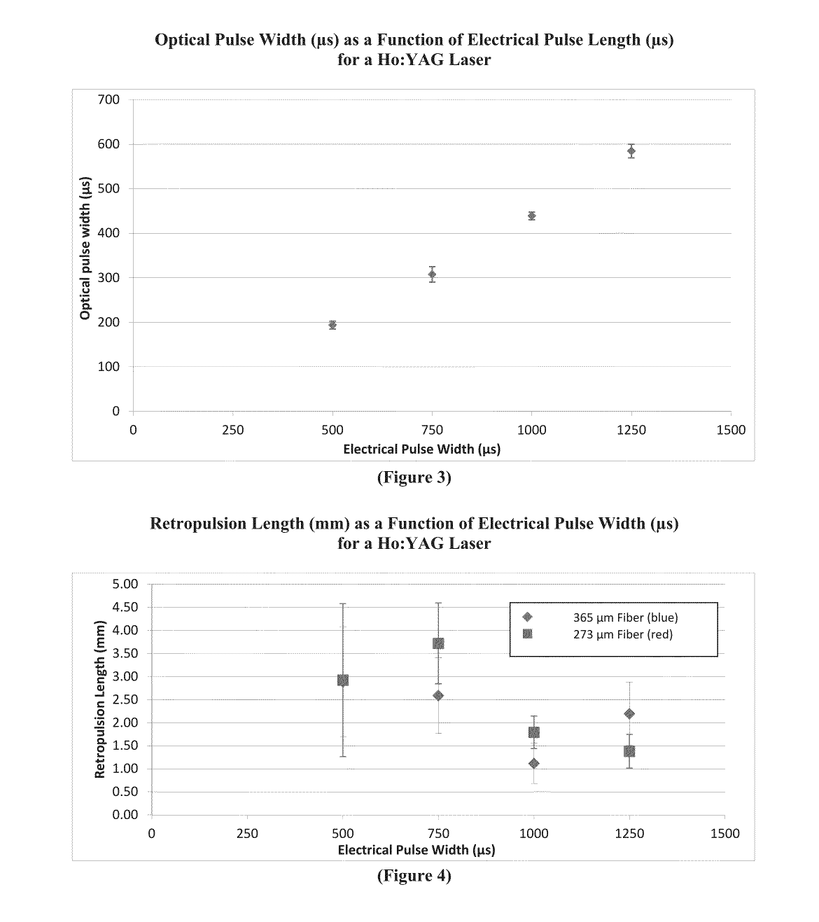

FIG. 3 illustrates the relationship between electrical pulse width and optical pulse width for a Ho:YAG laser. An electrical pulse width of 1000 μs correlates with an optical pulse width of 438.8 μs and an electrical pulse width of 1250 μs correlates with an optical pulse width of 584.4 μs.

In one aspect of the disclosed parameters, the ideal electrical pulse width at which the laser will be operated correlates with an optical pulse width that is less than 1000 μs. This is because the accepted maximum thermal relaxation time for tissue is 1000 μs. Any tissue exposure to energy longer than this maximum thermal relaxation time allows heat to build up in the tissue and results in unintended thermal effects such as increased coagulation within the tissue.

In another aspect of the disclosed parameters, the ideal electrical pulse widths at which the laser will be operated at are 1000 μs and 1250 μs. In FIGS. 4-6, a conventional Ho:YAG laser was tested with a 365 μm fiber (blue) and a 273 μm fiber (red) at a 1 J per pulse energy through a range of electrical pulse widths from 500 μs to 1250 μs. The data points demonstrate an effective electrical pulse width at 1000 μs and 1250 μs.

In another aspect of the disclosed parameters, the ideal optical pulse widths at which the laser will be operated at are 438.8 μs and 584.4 μs.

FIG. 4 illustrates the relationship between electrical pulse width and retropulsion length as the electrical pulse width is increased from 500 μs through 1250 μs. The retropulsion length increases up until an electrical pulse width of 750 μs and then decreases thereafter.

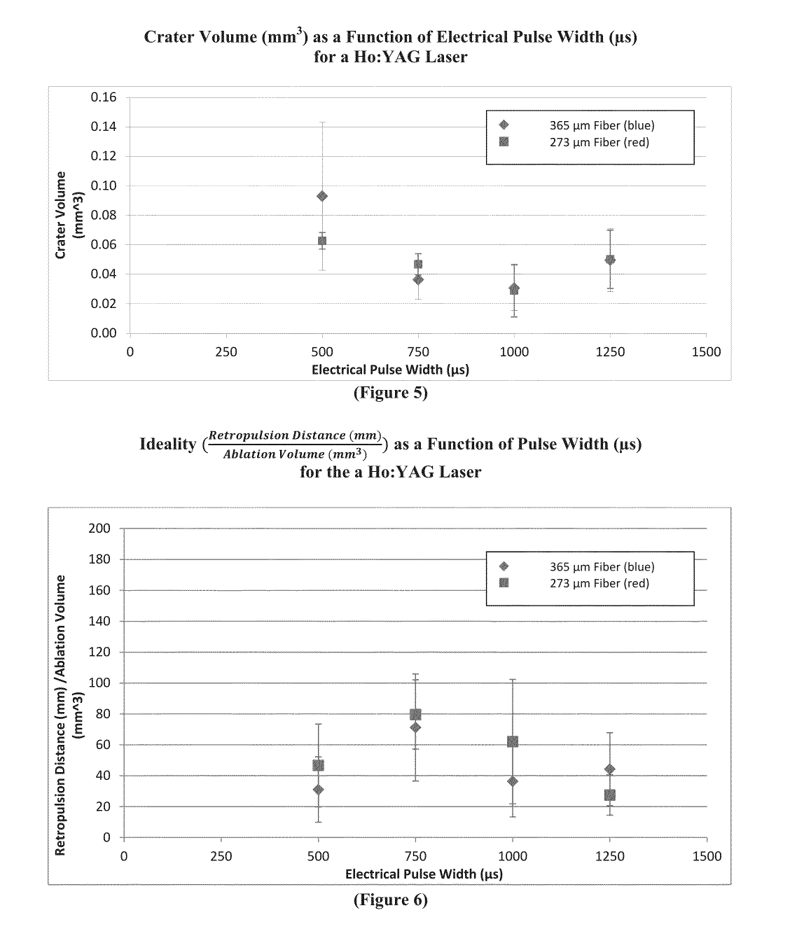

FIG. 5 illustrates the relationship between electrical pulse width and crater volume (ablation) as the electrical pulse width is increased from 500 μs through 1250 μs. Ablation decreases up until an electrical pulse width of 1000 μs and increases thereafter.

FIG. 6 illustrates the relationship between electrical pulse width and ideality as the electrical pulse width is increased from 500 μs through 1250 μs. Ideality increases through an electrical pulse width of 750 μs and decreases thereafter. Ideality is at its lowest at 1250 μs.

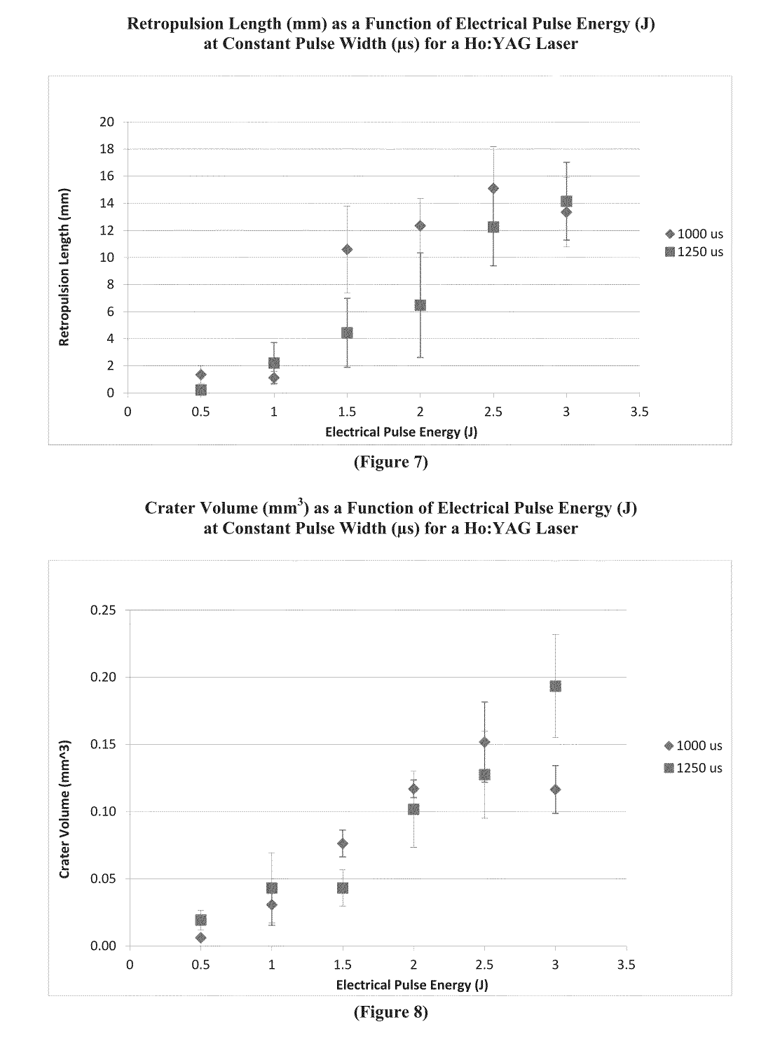

FIGS. 7-9 demonstrate the effect that increasing pulse energy has on retropulsion and ablation. In FIG. 7-9, a Ho:YAG laser was tested at both an electrical pulse width of 1000 μs and 1250 μs. While keeping the electrical pulse width constant at either of these two values, the range of electrical pulse energies was increased in increments of 0.5 J from 0.5 J to 3.0 J.

FIG. 7 illustrates the relationship between electrical pulse energy and retropulsion length as the electrical pulse energy was increased from 0.5 J through 3.0 J. The retropulsion length increased as electrical pulse energy was increased.

FIG. 8 illustrates the relationship between electrical pulse energy and crater volume (ablation) as the electrical pulse energy is increased from 0.5 J through 3.0 J. The crater volume increased as electrical pulse energy increased through 3.0 J.

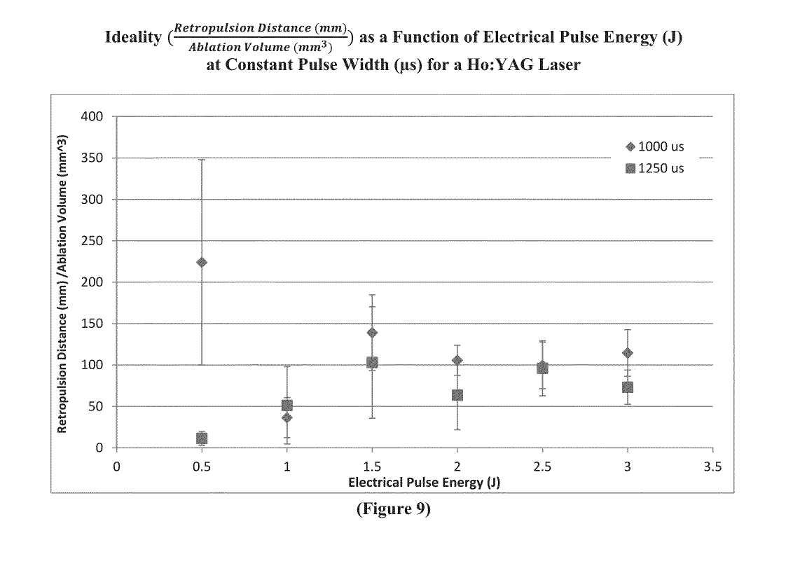

FIG. 9 illustrates the relationship between electrical pulse energy and ideality as the electrical pulse energy is increased from 0.5 J through 3.0 J. Ideality increased as electrical pulse energy increased and was lowest at an electrical pulse energy of 0.5 J. Although, as seen in FIG. 6, the crater volume increased as electrical pulse energy increased, the magnitude of increase in retropulsion length at higher electrical pulse energy rates offset the increase in crater volume as electrical pulse energy increased.

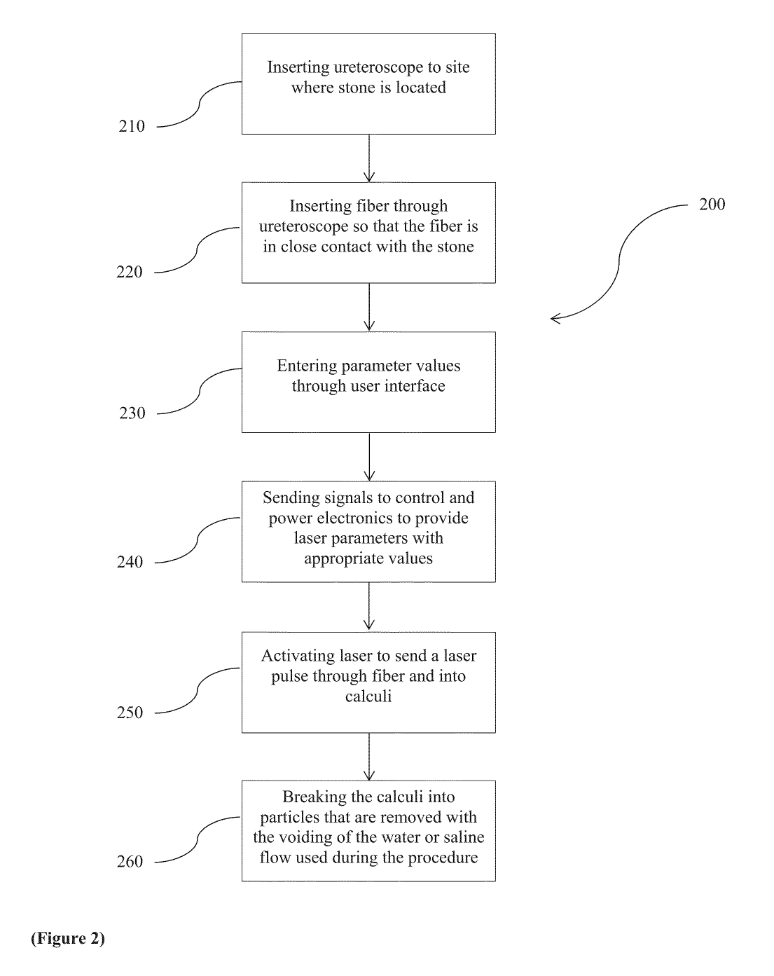

FIG. 2 is a flow chart of an embodiment of a laser system 200 configured to determine and identify the ideal values for laser parameters for calculi removal. First, at block 210 of FIG. 2, the ureteroscope 170 of FIG. 1 is inserted into the body to the site where the calculi 180 is located.

Next, at block 220 of FIG. 2, the fiber 160 of FIG. 1 is inserted through the ureteroscope 170 so that the fiber is near the calculi 180, with at least a film of water between the surface of the fiber 160 and the calculi 180.

Next, at block 230 of FIG. 2, parameter values are entered into user interface 140 of FIG. 1 to attain ideal values for electrical pulse width or optical pulse width. In one embodiment, the electric pulse width would be set at either 1000 μs or 1250 μs. In another embodiment, the optical pulse width would be set at either 438.8 μs or 584.4 μs.

Next, at block 240 of FIG. 2, the user interface 140 of FIG. 1 sends signals to the control electronics 130 and power electronics 120 to provide the ideal values for the laser parameters necessary to operate the laser 150.

Next, at block 250 of FIG. 2, the laser 150 of FIG. 1 is activated to send a laser pulse through the fiber 160 and into the calculi 180.

Next, at block 260 of FIG. 2, the calculi 180 is broken into fine particles that are voided with the water or saline flow used during the procedure.

While particular elements, embodiments, and applications of the present invention have been shown and described, it is understood that the invention is not limited thereto because modifications may be made by those skilled in the art, particularly in light of the foregoing teaching. It is therefore contemplated by the appended claims to cover such modifications and incorporate those features which come within the spirit and scope of the invention.

1. An apparatus for fragmenting calculi comprising:
a source of laser pulses,
an optical fiber having a distal end configured to be in close proximity with said calculi, and a proximal end that is configured to receive laser pulses from said source of laser pulses when said optical fiber is operatively engaged with said source of laser pulses, and
said source of laser pulses is configured to specifically generate laser pulses with an optical pulse width between 400 μs and 600 μs.

2. The apparatus of claim 1 wherein said optical fiber used has a 365 μm fiber.

3. The apparatus of claim 1 wherein said optical fiber used has a 273 μm fiber.

4. The apparatus of claim 1 wherein said source of laser pulses is a Ho:YAG laser.

5. The apparatus of claim 1 wherein said laser pulse has an optical pulse width between 600 μs and 1000 μs.

6. The apparatus of claim 1 wherein said laser pulse has an optical pulse width of 438.8 μs.

7. The apparatus of claim 1 wherein said laser pulse has an optical pulse width of 584.4 μs.

8. The apparatus of claim 1 wherein said laser pulse has an electrical pulse width at least 1000 μs.

9. The apparatus of claim 1 wherein said laser pulse has an electrical pulse width of 1000 μs.

10. An apparatus of claim 1 wherein said laser pulse has an electrical pulse width of 1250 μs.

11. A method for fragmenting calculi, the method comprising:
providing a source of laser pulses,
providing an optical fiber having a distal end configured to be in close proximity with said calculi, and a proximal end that is configured to receive laser pulses from said source of laser pulses when said optical fiber is operatively engaged with said source of laser pulses, and
calibrating said source of laser pulses to specifically generate laser pulses with an optical pulse width between 400 μs and 600 μs.

12. The method of claim 11 wherein said optical fiber used has a 365 μm fiber.

13. The method of claim 11 wherein said optical fiber used has a 273 μm fiber.

14. The method of claim 11 wherein said source of laser pulses is a Ho:YAG laser.

15. The method of claim 11 wherein said laser pulse has an optical pulse width between 600 μs and 1000 μs.

16. The method of claim 11 wherein said laser pulse has an optical pulse width of 438.8 μs.

17. The method of claim 11 wherein said laser pulse has an optical pulse width of 584.4 μs.

18. The method of claim 11 wherein said laser pulse has an electrical pulse width at least 1000 μs.

19. The method of claim 11 wherein said laser pulse has an electrical pulse width of 1000 μs.

20. The method of claim 11 wherein said laser pulse has an electrical pulse width of 1250 μs.
